(19) United States
(12) Patent Application Publication
(10) Pub. No.: US 2022/0409829 A1
(43) Pub. Date: Dec. 29, 2022

(54) ANTIVIRAL THAT DESTROYS DNA AND/OR RNA OF VIRUSES AND VIROIDS

(71) Applicant: Abdullah Al Mutairi, Al Majma'ah (SA)

(72) Inventor: Abdullah Al Mutairi, Al Majma'ah (SA)

(21) Appl. No.: 17/356,430

(22) Filed: Jun. 23, 2021

(51) Int. Cl. A61M 15/00; A61K 9/00; A61K 33/00; A61K 45/06; C01B 21/22

(52) U.S. Cl. CPC A61M 15/004; A61K 9/0073; A61K 33/00; A61K 45/06; C01B 21/22; A61M 2202/0283; A61M 2202/0208

(57) ABSTRACT

A system includes a charger and a cracker. An antiviral may be delivered through the charger. The cracker may include a first section and a second section to control the flow of the antiviral from the charger. The first section may receive the charger. The first section and the second section may be coupled together by using screw threads on the first and second sections. The cracker is configured to regulate the flow of antiviral into the user. The antiviral within the charger may include oxygen (O2) and nitrous oxide (N2O). Specifically, the antiviral may a mixture including 75% N2O and 25% O2 that inactivates the DNA and/or RNA of viruses, viroids, and germs.

### CROSS-REFERENCE TO RELATED APPLICATIONS

This application claims the benefit of U.S. Provisional Patent Application No. 63/014,257, filed Apr. 23, 2020, the entire contents of which are incorporated by reference.

### BACKGROUND

Viruses are found in almost every ecosystem on earth and are the most numerous types of biological entities. With millions of types of viruses found in the environment, only around 5,000 virus species have actually been described in detail. The millions of types of viruses are not limited in growth and many of these viruses may mutate to generate new strains of an existing virus, with each virus and/or mutation requiring a unique, separate vaccine. To address the effects of viruses, such as mild sickness to extreme sickness or death, many have turned to vaccines and others have turned to broad-spectrum antiviral drugs (BSA) that inhibit viral proteins, or target host cell proteins and processes exploited by the virus during infection.

Viruses alone are not a living organism They do not move, do not have energy, do not reproduce on their own, and do not have their own metabolic processes. A virus is inert unless it attaches to a living host, like the walls of living cells, so it can copy its DNA, reproduce and spread As long as the DNA is valid, it is capable of surviving in the same lineage or circulating in a new line viruses that are able to develop and integrate with other genes in the human body, which leads to new strains capable of overcoming acquired resistance to the body from previously received vaccines. As such, updating vaccines for each virus that evolves and overcomes previously discovered vaccines. In the future, it may be faster more deadly and threaten humanity, we must be prepared for any mysterious virus to ensure the safety of humanity. The radical solution is to destroy DNA or/and RNA of viruses and viroids, without damaging the human DNA.

### BRIEF DESCRIPTION OF THE DRAWINGS

The present description will be understood more fully when viewed in conjunction with the accompanying drawings of various examples of an antiviral that destroys DNA and/or RNA of viruses and viroids. The description is not meant to limit an antiviral that destroys DNA and/or RNA of viruses and viroids to the specific examples. Rather, the specific examples depicted and described are provided for explanation and understanding of an antiviral that destroys DNA and/or RNA of viruses and viroids. Throughout the description, the drawings may be referred to as drawings, figures, and/or FIGs.

FIG. 1 illustrates a side perspective view of a charger and cracker system, according to an embodiment.

FIG. 2 illustrates an antiviral, according to an embodiment.

FIG. 3 illustrates a diagram showing a method of producing nitrous oxide (N2O), according to an embodiment.

FIG. 4 illustrates a graph for relative onset effect of various gases, according to an embodiment.

FIG. 5 illustrates a table with the characteristics of various gases.

FIG. 6 illustrates a flowchart of a method of administering an antiviral.

FIG. 7 illustrates a diagram depicting an overview of viral infections in the human body.

### DETAILED DESCRIPTION

An antiviral that destroys DNA and/or RNA of viruses and viroids as disclosed herein will become better understood through a review of the following detailed description in conjunction with the figures. The detailed description and figures provide merely examples of the various embodiments of an antiviral that destroys DNA and/or RNA of viruses and viroids. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

Viruses are prevalent and are part of human life. With millions of types of viruses found in the environment, only around 5,000 virus species have actually been described in detail. To address some of these viruses, scientists have created vaccines, which are created from a weakened form or dead form of a specific virus. When vaccines are introduced into an individual, the weakened or dead form of the virus induces an immune response from the individual. This immune response leads to antibodies that protect the individual from the virus. Specifically, if the individual comes into contact with the virus and it enters the body, then the antibodies created from the vaccine attack the virus, protecting the individual. Other approaches to combating viruses include broad-spectrum (BSA) antiviral drugs. BSA drugs work by inhibiting viral proteins or by targeting host cell proteins and processes exploited by the virus during infection. In addition, many individuals may not want to receive a vaccine or BSA drugs and may want their bodies to independently create antibodies when coming in contact with a virus. In a similar manner to the vaccines, the body will create antibodies in response to the virus interacting with the body's DNA, but in this case, the individuals will more than likely suffer from the effects of the virus.

The millions of types of viruses are not limited and many of these viruses may mutate to generate new strains of existing viruses. While vaccines are helpful and have created safety for many, they also have many shortcomings. For example, each virus and each mutation of a given virus may require its own, unique vaccine. In particular, viruses are able to develop and integrate with other genes in the human body, which leads to new strains capable of overcoming acquired resistance to the body from previously received vaccines. As such, updating vaccines for each virus that evolves and overcomes previously discovered vaccines, is required to ensure the safety of humanity. Trying to create a vaccine for every type of virus is cost-prohibitive and in reality, impossible. Consequently, vaccines are not the ultimate solution to combating the numerous effects of so many viruses. In addition, BSA drugs are also not antiviral drugs that destroy the target pathogen, and they may have adverse effects. Further, many individuals seek to live without vaccines or BSA drugs and choose to let a virus run its course. A lot of risks are involved when a virus enters a human being and each individual may react differently, with some having mild reactions and others having severe reactions. Treatments vary dramatically as well as opinions on viruses and treatments. This often arises from confusion around the current classification of viruses as microorganisms.

A charger and a cracker for holding antiviral that destroys DNA and/or RNA of viruses and viroids are disclosed herein that address at least some of the problems described above. The antivirus may be delivered through a charger that is suitable for human use, easy to use, inexpensive to manufacture, and less resource intensive. The charger may be cylindrical. The charger may be manufactured from a metal material, such as steel. The charger may comprise a first end that is enclosed and rounded. A second end of the charger may include a narrow tip. The charger may comprise walls of a thickness to withstand the pressure of the gases received therein. In addition, a cracker device may be coupled to the charger so as to regulate the release of gas from the charger.

The cracker may comprise a first section and a second section to control the flow of gas from the charger. The first section may receive the charger. The first section and the second section may be coupled together by using screw threads on the first and second sections or by any other securing mechanism. When the second section is coupled to the first section, a puncturing pin in the second section pierces the wall of the charger, thereby releasing the gas. The gas may then be released through one or more apertures on the second section. For example, the second section may comprise two apertures that are spaced so as to be placed below each nostril of an individual. This allows the gas contained within the charger to enter the individual. It should be noted that when gas is released from the charger, it is extremely cold and can cause damage, such as frostbite, to the lips, tongue, throat, and lungs. Accordingly, the cracker regulates the gas and allows it to warm before it is inhaled by the individual.

The antiviral within the charger may include oxygen (O2) and nitrous oxide (N2O). The antiviral may be of a mixture including 75% N2O and 25% O2. However, N2O and O2 may be administered in a different ratio. In addition, the antivirus in the charger may also comprise antiviral, bacteriostatic, analgesic, anxiolytic, and/or antidepressant. Because it is a gas, there may be no maximum dose. Nitrous oxide has been used for over a hundred years as an anesthetic and analgesic. Typically, nitrous oxide is given to individuals via automated machines that are limited in number and only used by healthcare institutions Such machines may include an automated relative analgesia machine, with an anesthetic vaporizer and a medical ventilator, that delivers a precisely dosed and breath-actuated flow of nitrous oxide mixed with oxygen in a 2:1 ratio. However, with the charger and cracker an individual may conveniently carry and administer the antiviral into their body or others. The antiviral may be delivered to individuals through the automated machines found in hospitals and other healthcare entities. The antiviral is an antiviral for general viruses. The antiviral may be inhaled, and its properties damage the DNA and/or RNA in viruses, viroids, and germs to prevent them from interacting with the host. The antiviral may be suitable for combating all or many viruses by attacking each virus's basic structural characteristics regardless of its classification and composition. Accordingly, the antiviral eliminates viruses and microbes at their roots by destroying their DNA.

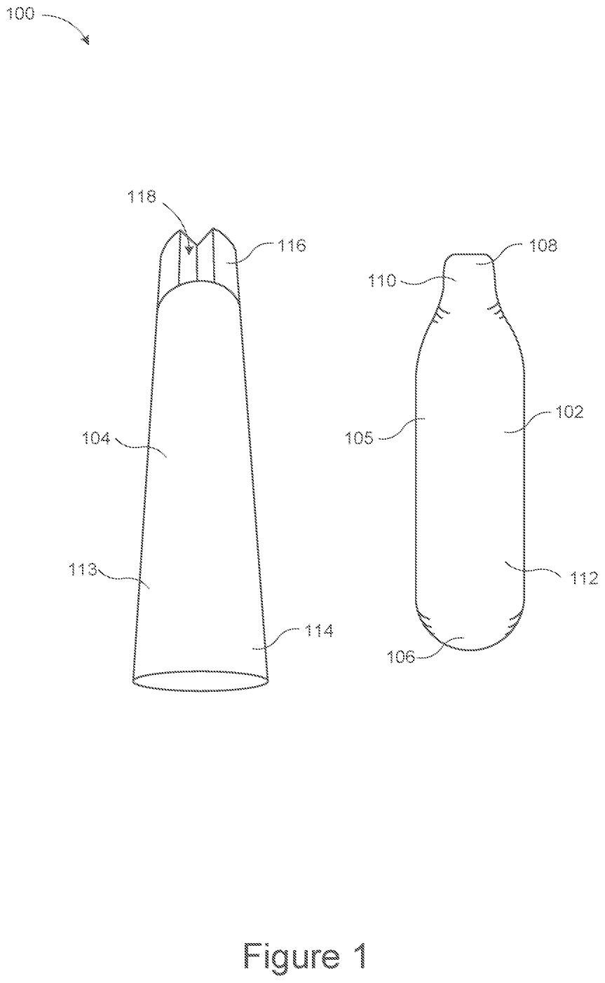

FIG. 1 illustrates a side perspective view of a charger and cracker system 100, according to an embodiment. The charger and cracker system 100 includes a cylindrical charger 102 positioned within a cracker 104. The charger and cracker system 100 may allow a user access to an antiviral at a lower cost and be easily administered.

The antiviral may be delivered through the charger 102. The charger 102 may be suitable for human use, easy to use, inexpensive to manufacture, and less resource-intensive than other options of delivering gas to a user. The charger 102 may include a first housing 105 that is filled with a compressed gas antiviral. The charger 102 may be placed within the cracker 104 (a container that can spray or extrude the compressed gas from the charger 102), where the antiviral may be released to a user. The charger 102 may be a steel cylinder filled with N2O that may be release via the cracker. The first housing 105 may be cylindrical; however, the shape of the charger 102 is not so limited and may include any other shape, such as rectangular. In one embodiment, the first housing 105 may include a first length (e.g., about 6.5 cm (2.55 inches) long) and a first width (e.g., 1.8 cm (0.7 inches) wide). Further, the charger 102 may also be in a range of 6.5 cm-7.5 cm long and 1.8 cm-2.8 cm wide. The charger 102 may be manufactured from a metal material, such as steel. Other materials may be used for the charger 102, such as plastics. The first housing 105 may include a first end 106 that is enclosed and rounded. In one embodiment, the tip 110 may include a foil cover that may be punctured to release the antiviral. A second end 108 of the charger may include a narrow, elongated neck or tip 110. The charger 102 may include sidewalls 112 of a thickness to withstand the pressure of the gases received therein. For example, in one embodiment, the charger's walls 112 are about 2 mm (about 1/16 inch) thick to withstand the pressure of the gas contained within. In other embodiments, the charger's wall 112 is in a range from 1.5 mm-2.5 mm thick. The interior of the charger 102 may have an interior volume that is 10 cm³ (about 0.6 in³), which may contain a dose of antiviral (e.g., 4 g of N2O under medium pressure). In other embodiments, the volume of the interior is in a range from 9 cm³-11 cm³. The charger 102, in some embodiments, may maintain a max pressure of 15 pounds per square inch (100 kPa) and deliver 1.62 liters of nitrous oxide gas. Such pressure may equal the pressure of the lungs to enhance the effectiveness of the antiviral administration. The amount of antiviral in the charger 102 may be of a quantity for one-time use by a user or of a quantity that allows numerous doses. In one embodiment, the antiviral within the charger 102 may include from 1 g to 22 g N2O from 50% to 80% and O2 from 20% to 50%. In other embodiments, the antiviral within the first housing 105 of the charger 102 may include N2O from 50% to 80% and O2 from 20% to 60%, with both N2O and O2 being combined to equal 100% or less than 100% of the antiviral. While the charger 102 is discussed above as having specific lengths, widths, and wall thicknesses, it will be appreciated that the charger 102 may come in numerous sizes, shapes, and wall thickness. The flow of antivirus out of the charger 102 may be regulated by the cracker 104 so that a user may inhale the antiviral.

In addition, the cracker device 104 may include a second housing 113, which may be coupled to and house the charger 102 so as to regulate the release of antivirus from the charger 102. Specifically, a user may place the charger 102 within the second housing 113 to regulate the release of the antivirus for inhalation. The second housing 113 may include a first section 114 and a second section 116 to control the flow of gas from the charger 102. The first section 114 may receive and house the charger 102. The first section 114 and the second section 116 may be coupled together by using screw threads on the first and second sections 114, 116, or by any other securing mechanism. When the second section 116 is coupled to the first section 114, a punctuter (e.g., a puncturing pin) in the second section 116 pierces the wall 112 of the charger 102, thereby releasing the antivirus. The antiviral may be released through one or more apertures 118 on the second section 116. In some embodiments, the second section 116 may include two apertures that are spaced so as to be placed below each nostril of an individual. This allows the antiviral contained in the charger 102 to be released and enter the individual, damaging the DNA and/or RNA of viruses, viroids, and germs. It will be appreciated that when gas is released from the charger 102 it is extremely cold and cause damage, such as frostbite, to the lips, tongue, throat, and lungs. Accordingly, the cracker 104 regulates the gas and allows it to warm before it is inhaled by the individual.

While the charger and cracker system 100 is described above, it will be noted that other methods of administering the antiviral may be used, such as metered-dose inhalers, inhalation sedation, inhalers, inhalation solutions, nebulizer with saline, or any other method or device.

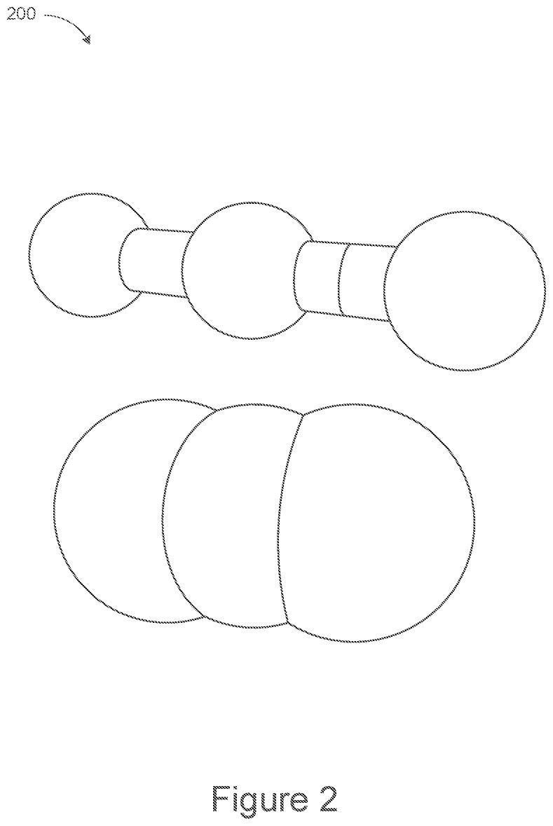

FIG. 2 illustrates an antiviral 200, according to an embodiment. The antiviral 200 includes nitrous oxide (N2O) and oxygen (O2). The antiviral 200 may be inhaled by a user to damage RNA and/or DNA found in viruses, viroids, and germs.

The antiviral 200 may be inserted into the charger and cracker system 100. The antiviral may include O2 and N2O. The antiviral may a mixture including 75% N2O and 25% O2. In some embodiments, N2O and O2 may be administered in a different ratio, such as 1:1, 2:1, 3:1, or to a max of 4:1. In some embodiments, the antiviral in the charger may also include antiviral, bacteriostatic, analgesic, anxiolytic, and/or antidepressant. The antiviral 200 may be a strong oxide having several properties for damaging the RNA and/or DNA in a virus or viroids. These properties may include the following: reductive dissolution; hydrolysis and dissolution; oxides that react with acids and/or bases; protein; nitrous-oxide reductase; and nitrous oxide, which oxidizes an active form of cobalamin (i.e., vitamin B12), making it inert.

The adverse effects of N2O—except for nausea, vomiting, and neuroapoptosis—may be due to the inactivation of cobalamin. Cobalamin is an important coenzyme in the conversion of homocysteine to methionine. Methionine uses folate to synthesize myelin, DNA, and RNA. N2O inhibition of cobalamin can lead to impaired DNA synthesis and reduced levels of methionine, possibly resulting in impaired metabolic pathways. N2O irreversibly oxidizes the cobalt atom of vitamin B12 and thereby reduces the activity of B12-dependent enzymes such as methionine and thymidylate synthetases. This may be the mechanism for toxicity because these enzymes are vital in the synthesis of myelin and nucleic acids. It is the impaired DNA synthesis that can damage viruses. Megaloblastic changes in bone marrow are observed following exposure to anesthetic concentrations for 24 hours, and agranulocytosis is apparent after 4 days of exposure to causing damage to fragile viruses DNA from less than 2 kb of single-stranded DNA to over 375 kb that have only 42 proteins on average coded in their respective genomes.

The human genome size is 3,234.830 kp, with 1 to 3 billion proteins. The antiviral 200 may react with the nitrogenous bases. In the biological sciences, nitrogenous bases are increasingly termed nucleobases because of their role in nucleic acids—their flat shape is particularly important when considering their roles as the building blocks of DNA and RNA. A set of five nitrogenous bases is used in the construction of nucleotides, which in turn build up nucleic acids like DNA and RNA. These nitrogenous bases are adenine (A), uracil (U), guanine (G), thymine (T), and cytosine (C). Thymine and uracil are distinguished by merely the presence or absence of a methyl group on the fifth carbon (C5) of these heterocyclic six-membered rings and with Amino acids, are the main building block of protein and peptides. The antiviral 200 affects the protein and DNA and/or RNA of viruses and viroids without damaging human DNA.

Microorganisms may include viroids that consist of DNA, RNA, or both; prions that consist of protein; and viruses that consist of DNA, RNA, or both, protein, and in some cases, an outside envelope of lipids. Vaccines on the market seek to increase antibodies in a user to combat viruses. However, vaccines are not always effective and do not destroy the DNA or RNA in viruses. On the other hand, the antiviral 200 isolates, destroys, and dismantles the DNA or RNA of any virus, preventing the virus from adhering to cell walls within the human body and releasing DNA or RNA that can cause harm. In one example, the antiviral may not be not limited to including N2O and may include a binary compound of oxygen and nitrogen, or a mixture of such compounds. For example, charge-neutral compounds may be used, such as the following: nitric oxide (NO), nitrogen(II) oxide, nitrogen monoxide, nitrogen dioxide (NO2), nitrogen(IV) oxide, nitrogen trioxide (NO3), nitrate radical, nitrous oxide (N2O), nitrogen(0,II) oxide, dinitrogen dioxide (N2O2), nitrogen(II) oxide dimer, dinitrogen trioxide (N2O3), nitrogen(II,IV) oxide, dinitrogen tetroxide (N2O4), nitrogen(IV) oxide dimer, dinitrogen pentoxide (N2O5), nitrogen(V) oxide, nitronium nitrate [NO2]+[NO3]−, nitrosylazide (N4O), nitrogen(−I,0,I,II) oxide, oxatetrazole (N4O), trinitramide (N(NO), or nitrogen(0,IV) oxide.

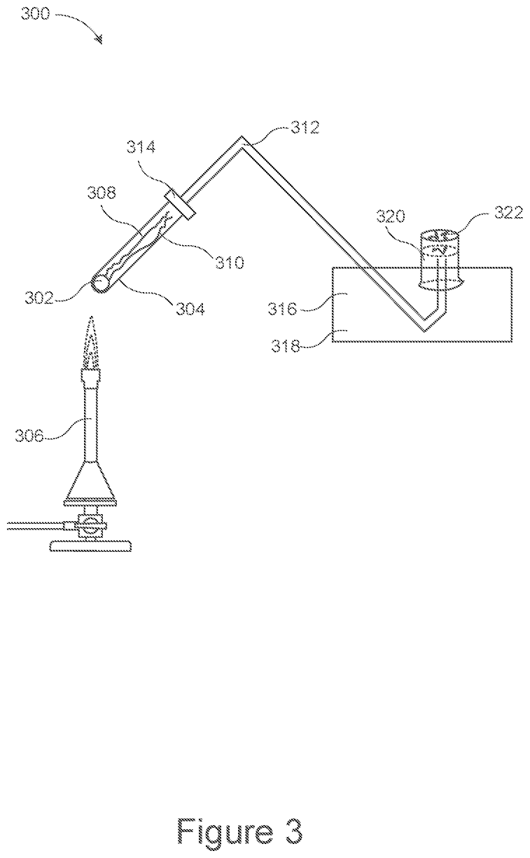

FIG. 3 illustrates a system 300 showing a method of producing nitrous oxide (N2O), according to an embodiment. The system 300 includes steps to produce N2O. The system 300 allows a user to produce the antiviral 200.

The system 300 illustrates the production of N2O through one method. To produce N2O, a user may place ammonium nitrate 302 into a test tube 304. A Bunsen burner 306 may then, in some embodiments, heat the test tube 304 with the ammonium nitrate 302 to a temperature of 200 degrees Celsius. From the heat, N2O 308 and water vapor 310 are produced and leave the test tube 304 via a pipe 312, which is coupled to the test tube 304 by a test tube cap 314. The N2O 308 and water vapor 310 then travel through the pipe 312. The pipe 312 descends into a housing 316 that has hot water 318 and then ascends out of the water into a beaker 320. In one example, hot water 318 may be used because N2O is prone to dissolve in cold water. Through this process, pure N2O 322 is deposited into the beaker 320. Accordingly, nitrous oxide production via industrial methods involves heating of ammonium nitrate at about 200-250 C, which decomposes into nitrous oxide and water vapor as follows: NH4 NO3→2 H2O+N2O.

Other methods of producing the antivirus 200 may include laboratory methods involving heating a mixture of sodium nitrate and ammonium sulfate: 2 NaNO3+(NH4)2SO4→Na2SO4+2 N2O+4 H2O. Another method may involve the reaction of urea, nitric acid and sulfuric acid as follows:

2(NH2)2CO+2HNO3+H2SO4→2N2O+2CO2+(NH4)2SO4+2H2O

2NH3+2O2→N2O+3H2O

NH3OHCl+NaNO2→N2O+NaCl+2H2O

2HNO3+8HCl+4SnCl2→5H2O+4SnCl4+N2O

In addition, another method may involve hyponitrous acid, which decomposes to N2O and water with a half-life of 16 days at 25° C. at pH 1-3 as follows: H2N2O2→H2O+N2O.

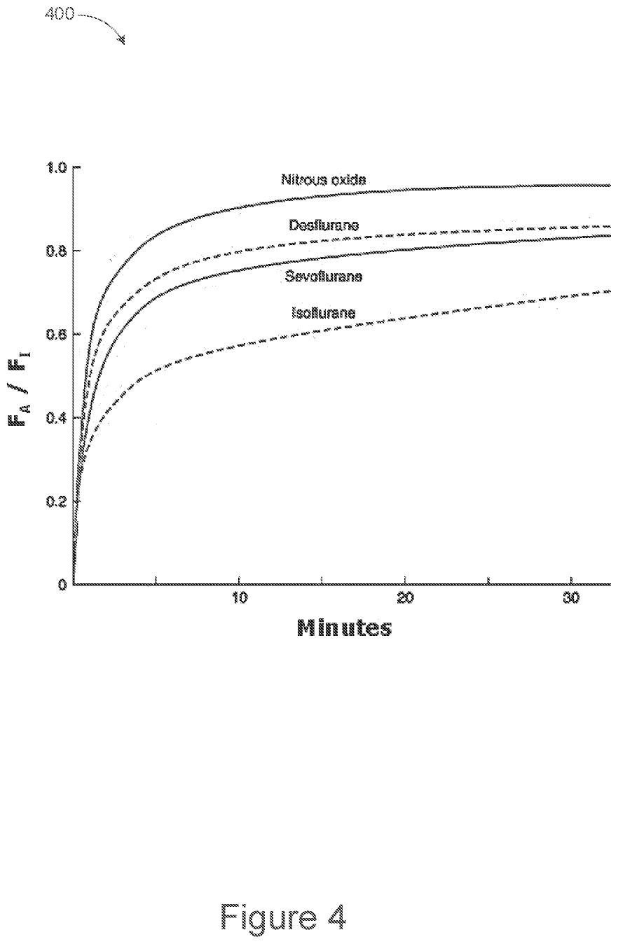

FIG. 4 illustrates a graph 400 for relative onset effect of various gases, according to an embodiment. The graph 400 shows the increase in the rate of Fa (alveolar concentration)/Fi (inspired concentration) ratio with certain gases, including nitrous oxide, over time.

The graph 400 shows that inhalation agents are respiratory depressants, and their influence on ventilatory response to hypoxemia is greater than that for hypercapnia. When gas tensions throughout body tissues equilibrate, the Fi (inspired concentration or gas tension) will equal that in the Fa (alveolar concentration). The graph 400 illustrates that nitrous oxide achieves approximately 90% equilibration within 10 minutes. For each gas illustrated in the graph 400, the speed of onset correlates with partition coefficients. Concentrations <0.5 minimum alveolar concentration (MAC) have minimal influence on hypercapnic drive, but the dose response becomes more significant at higher concentrations, leading to apnea at concentrations of 1.5 to 2.0 MAC. However, in contrast, as little as 0.1 MAC produces a 50% to 70% reduction in ventilatory response to hypoxemia. Accordingly, with regard to N2O, there are few side effects for human use, with short-term use. However, with long-term use anemia or numbness may occur. When using the antiviral 200 and to avoid long-term issues to the human body, in some embodiments, 21% oxygen may be used.

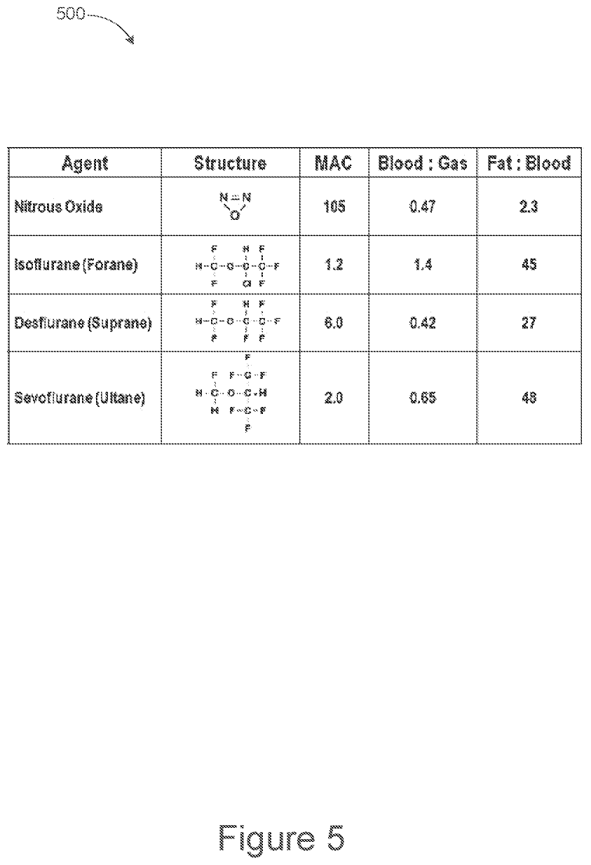

FIG. 5 illustrates a table 500 with characteristics of various gases. The table 500 includes specific types of gases with their molecular structure, MAC, blood:gas, and fat:blood. For example, the table shows the molecular structure for nitrous oxide (N2O), its MAC, blood:gas, and fat:blood. Specifically, N2O has a minimum alveolar concentration of 105% and a blood/gas partition coefficient of 0.46.

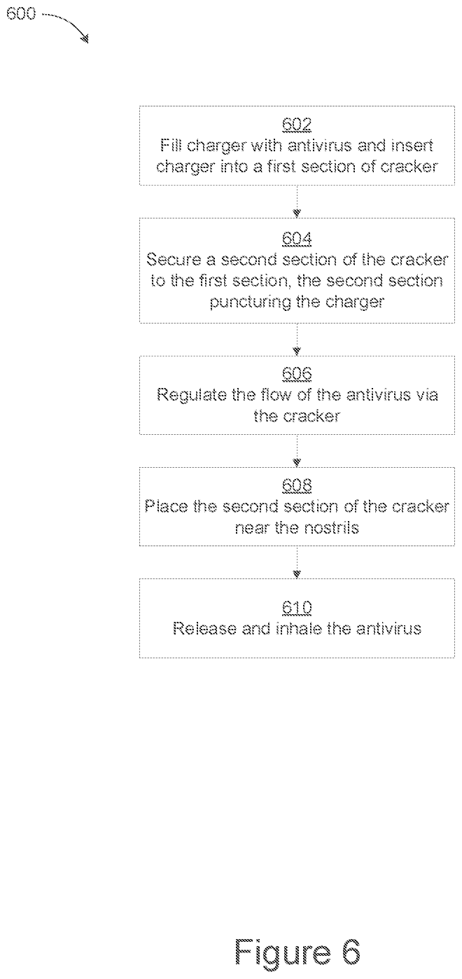

FIG. 6 illustrates a flowchart of a method of administering an antiviral 600. The method 600 includes using a charger and cracker to deliver the antivirus 200 to a user. The method 600 allows users to administer the antivirus easily and effectively to themselves or others.

The method 600 includes, at step 602, inserting a charger filled with an antiviral into a first section of a cracker. At step 604, a user places and secures a second section of the cracker to the first section, the second section punctures the charger. The user then may regulate the flow of the antivirus via the cracker at step 606. Then, at step 608, the user places the cracker, which includes one or more apertures on the second section, near their nostrils. At step 610, flow of the antivirus is then released, having enough time to warm without causing damage to the user, and the user inhales the antiviral. Once the antivirus enters the user, it can cause damage to any viruses within the user.

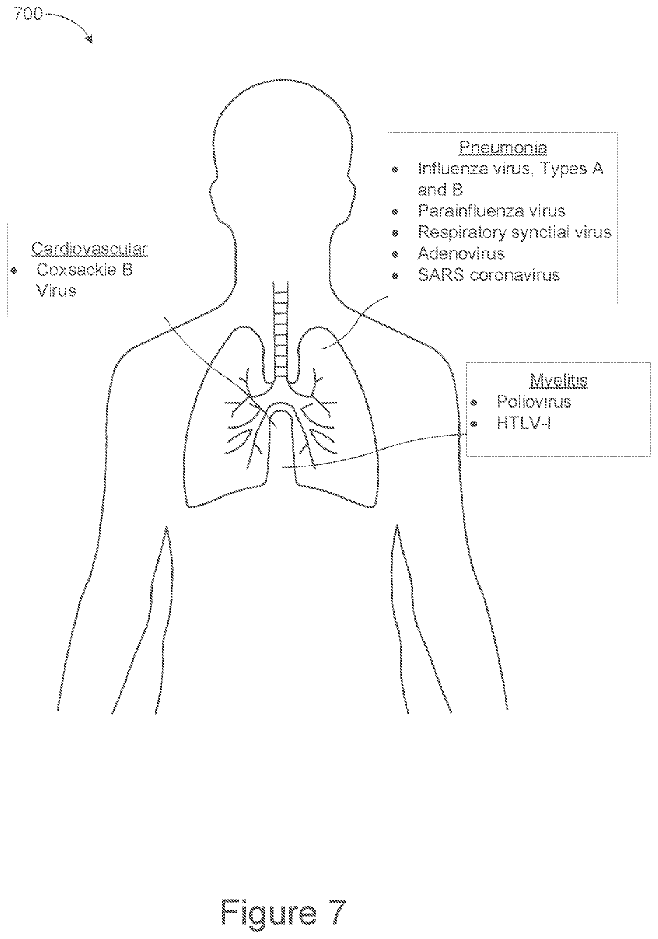

FIG. 7 illustrates a diagram 700 depicting an overview of viral infections in the human body. The human body may contract many viruses, both internally and externally. The antiviral 200 destroys DNA or RNA, or both, in all viruses, viroids, and germs. Prevention of the pathogens may be destroyed by nitrous oxide (N2O) within the antiviral.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower or equal in scope to the original claims, are to be considered within the subject matter of the examples described herein.

1. A method comprising:
filling a first housing of a charger with an antiviral, the first housing comprising:
a first end that is enclosed and rounded;
a second end, opposite the first end, comprising an elongated neck configured to release the antiviral; and
one or more sidewalls extending between the first end and the second end;
wherein:
the antiviral is a mixture comprising at least nitrous oxide (N2O) and oxygen (O2);
the N2O forming between 50% and 80% of the mixture; and
the O2 forming between 20% and 50% of the antiviral, wherein the N2O and the O2 combine to form up to 100% of the mixture;
inserting the charger filled with the antivirus into a cylindrical cracker comprising a second housing, the second housing comprising:
a first section configured to receive the first end of the charger; and
a second section comprising one or more apertures to release the antiviral from the charger, wherein the second section is configured to connect to the first section to form a container to enclose the charger; and
coupling the second section of the cylindrical cracker to the first section of the cylindrical cracker;
puncturing the charger when the second section is coupled to the first section, the second section comprising a puncturer coupled thereto to puncture the charger;
regulating the release of antiviral from the charger via the cylindrical cracker;
positioning the one or more apertures on the second section of the cylindrical cracker near one or more nostrils of a user; and
discharging, by the cylindrical cracker, the antiviral for inhalation into a body of the user to damage DNA and/or RNA of a virus or viroid and prevent adhesion to cell walls within the body.

2. The method of claim 1, wherein:
a length of the first housing is between 6.5 centimeters (cm) long and 7.5 cm long; and
a width of the first housing is between 1.8 cm wide and 2.8 cm wide.

3. The method of claim 1, wherein the one or more sidewalls are between 1.5 millimeters (mm) thick and 2.5 mm thick to withstand a pressure of the of the N2O and O2 within the first housing.

4. The method of claim 1, wherein the charger comprises an interior volume between 9 cm³ and 11 cm³.

5. The method of claim 1, wherein the charger includes a single dose of antiviral that comprises at least 4 grams (g) of N2O.

6. The method of claim 1, wherein the N2O and O2 within the charger comprises a pressure equal to a pressure of lungs of the user.

7. The method of claim 1, wherein the charger comprises a single dose of antiviral that is dischargeable.

8. The method of claim 1, wherein the charger comprises multiple doses of the antiviral:
that are dischargeable for multiple uses by the user, or
multiple uses for many users.

9. The method of claim 1, wherein the antiviral comprises from 1 gram (g) to 22 g of N2O.

10. The method of claim 1, wherein the antiviral comprises one or more of a bacteriostatic, an analgesic, an anxiolytic, or an antidepressant.

11. A device comprising:
a charger comprising a first housing with an antiviral that damages DNA and/or RNA of a virus or viroid therein, the first housing comprising:
a first end that is enclosed and rounded; and
a second end, opposite the first end, comprising an elongated neck configured to release the antiviral;
a cracker comprising a second housing to receive the charger, the cracker comprising:
a first section configured to receive the first end of the first housing, and
a second section configured to receive the second end of the first housing, wherein:
the second section is configured to couple to the first section via a fastener; and
the second section comprises one or more apertures, where the antiviral is released;
wherein when the second section is coupled to the first section, the second section punctures the first housing of the charger to release the antivirus into the cracker.

12. The device of claim 11, wherein:
the antiviral comprises from 1 g-22 g of nitrous oxide (N2O); and
N2O from 50% to 80% and oxygen (O2) from 20% to 50%, with both the N2O and the O2 being combined to equal up to 100% of the antivirus.

13. The device of claim 11, wherein the antiviral comprises a binary compound of oxygen and nitrogen, or a mixture of such compounds.

14. The device of claim 13, wherein the mixture of such compounds may comprise one or more of nitric oxide (NO), nitrogen(II) oxide, nitrogen monoxide, nitrogen dioxide (NO2), nitrogen(IV) oxide, nitrogen trioxide (NO3), nitrate radical, nitrous oxide (N2O), nitrogen(0,II) oxide, dinitrogen dioxide (N2O2), nitrogen(II) oxide dimer, dinitrogen trioxide (N2O3), nitrogen(II,IV) oxide, dinitrogen tetroxide (N2O4), nitrogen(IV) oxide dimer, dinitrogen pentoxide (N2O5), nitrogen(V) oxide, nitronium nitrate [NO2]+[NO3]−, nitrosylazide (N4O), nitrogen(—I,0,I,II) oxide, oxatetrazole (N4O), trinitramide (N(NO), or nitrogen(0,IV) oxide.

15. The device of claim 11, wherein the antivirus comprises one or more of a bacteriostatic, an analgesic, an anxiolytic, or an antidepressant.

16. The device of claim 11, wherein when the second section is secured to the first section, the second section punctures the charger to release the antiviral.

17. A method comprising:
filling a first housing of a charger with an antivirus comprising nitrous oxide (N2O) and oxygen (O2), the first housing comprising:
a first end that is enclosed and rounded,
a second end, opposite the first end, comprising an elongated neck configured to release the antiviral, and
one or more sidewalls extending between the first end and the second end;
inserting the charger filled with the antivirus into a second housing of a cylindrical cracker, the second housing comprising:
a first section configured to receive the first end of the first housing, and
a second section, separate from the first section, comprising one or more apertures, wherein the second section is configured to connect to the first section to form a container to enclose the charger;
coupling the second section of the cylindrical cracker to the first section of the cylindrical cracker; and
regulating the release of antivirus from the charger via the cylindrical cracker.

18. The method of claim 17, wherein the first housing comprises a length between 6.5 cm and 7.5 cm long and a width between 1.8 cm and 2.8 cm.

19. The method of claim 17, wherein the N2O is produced by a method comprising heating a mixture of sodium nitrate and ammonium sulfate.

20. The method of claim 17, wherein the N2O is produced by a method comprising:
placing ammonium nitrate into a container;
heating the ammonium nitrate between 200 degrees Celsius to 250 degrees Celsius;
collecting the N2O and water vapor produced from heating the ammonium nitrate; and
separating the N2O from the water vapor.
